(19) United States
(12) Patent Application Publication
(10) Pub. No.: US 2026/0077161 A1
(43) Pub. Date: Mar. 19, 2026

(54) CATHETER SYSTEMS AND HANDLES

(71) Applicant: yoR Labs, Inc., Beaverton, OR (US)

(72) Inventor: Dakota Pellegrino, Hillsboro, OR (US)

(21) Appl. No.: 19/109,045

(22) PCT Filed: Sep. 1, 2023
(86) PCT No.: PCT/US2023/073371

§ 371 Date: Mar. 5, 2025

Related U.S. Application Data
(60) Provisional application No. 63/374,743, filed on Sep. 6, 2022.

(51) Int. Cl. A61M 25/01; A61B 8/00; A61B 8/08; A61B 8/12; A61B 18/00; A61B 18/14; A61B 90/98

(52) U.S. Cl. CPC A61M 25/0136; A61B 8/12; A61B 8/4438; A61B 8/4444; A61B 8/467; A61B 8/565; A61B 18/1492; A61B 90/98; A61B 8/0883; A61B 8/0891; A61B 2018/00077; A61B 2018/00083; A61B 2018/00178; A61B 2018/00184; A61B 2018/00577; A61B 2018/00952; A61M 2205/3592; A61M 2205/6072; A61M 2209/084

(57) ABSTRACT

A catheter handle that includes a housing having a longitudinal axis, the housing having a distal end and a proximal end, a mechanical interface on the distal end for coupling to a catheter, an opening on the proximal end of the housing, an aperture extending from the opening into the housing; a first electrical interface positioned on the proximal end of the housing for electrically connecting to a cable; and a second electrical interface positioned at the distal end of the housing, the second electrical interface having a proximal side facing the aperture and distal side facing the opposite direction, the second electrical interface positioned to be accessible on the proximal side via the aperture, and accessible on the distal side to electrically connect to a catheter coupled to the catheter handle.

### INCORPORATION BY REFERENCE TO ANY PRIORITY APPLICATIONS

Any and all application for which a foreign or domestic priority claim is identified in the Application Data Sheet as filed with the present application are hereby incorporated by reference under 37 CFR 1.57. This disclosure is related to PCT Application PCT/US2023/073371, filed Sep. 1, 2023, and U.S. Provisional No. 63/374,743, filed on Sep. 6, 2022, both of which are incorporated by reference herein in their entirety.

### TECHNICAL FIELD

The invention relates to catheter medical systems. Specifically, the invention relates to a catheter devices for cardiac or vascular diagnostic and interventional procedures.

### BACKGROUND

Catheters are commonly used to access patient anatomy for medical reasons. Catheters are often exposed to biomaterials including bodily fluids, tissue, and pathogens. Accordingly, catheters are typically single-use devices to be disposed of after each use to prevent the transmission of biomaterials from one patient to another. Due to their single-use nature and to minimize costs, catheters are often constructed from inexpensive materials with simple mechanical elements for providing controls and lack sophisticated electrical components and control systems.

Instead of processing data itself, catheters transmit data, including imaging data, to separate sterilized equipment. The data is often complex and includes large data sets requiring the use of cables extending from the catheter to the data processing equipment. Some equipment is only configured to process certain types of data. Accordingly, operating rooms may comprise a plurality of processing equipment configured to process a certain category of data and a plurality of cables extending from the equipment to the catheters. Additionally, operating rooms are often small and have many care takers present during an operation. Thus, the plurality of cables presents a hazard within the operating rooms. Thus, there remains a need to simplify the operating room by removing physical tripping hazards and minimizing the number of unnecessary or redundant equipment.

### SUMMARY

Certain aspects of this invention are defined by the independent claims. The dependent claims concern optional features of some embodiments of the invention. The systems, methods, and devices described herein each have several aspects, no single one of which is solely responsible for its desirable attributes. Without limiting the scope of this disclosure, several non-limiting features will now be discussed briefly.

One innovation includes an a catheter handle, including a housing having a longitudinal axis, the housing having a distal end and a proximal end, a mechanical interface on the distal end for coupling to a catheter, an opening on the proximal end of the housing, an aperture extending from the opening into the housing; a first electrical interface positioned on the proximal end of the housing for electrically connecting to a cable; and a second electrical interface positioned at the distal end of the housing, the second electrical interface having a proximal side facing the aperture and distal side facing the opposite direction, the second electrical interface positioned to be accessible on the proximal side via the aperture, and accessible on the distal side to electrically connect to a catheter coupled to the catheter handle.

Various embodiments can include one or more additional features. In some embodiments, the first electrical interface includes a set of electrical contacts. In some embodiments, the first electrical interface is positioned around the opening. In some embodiments, the first electrical interface includes an insulator ring coupled to the housing and positioned around the opening, wherein first electrical interface includes a set of electrical contacts located in the insulator ring such that the insulator ring electrically insulates the set of electrical contacts from the housing. In some embodiments, the set of electrical contacts comprise one or more flat electrically conductive contact pads. In some embodiments, the set of electrical contacts comprise one or more pogo pins. In some embodiments, the set of electrical contacts are positioned in a circle on the proximal end of the handle. In some embodiments, the set of electrical contacts includes two or more electrical contacts. In some embodiments, the set of electrical contacts includes three, four, five, six, seven, eight, none, ten or more electrical contacts. In some embodiments, the catheter handle further comprises a set of electrical controls connected to the first electrical interface. In some embodiments, the set of electrical controls includes two of more electrical controls each connected to a separate electrical contact of the first electrical interface.

In some embodiments of a catheter handle, the second electrical interface includes a set of electrical contacts that extend from the proximal side of the second electrical interface to the distal side of the second electrical interface. In some embodiments, the set of electrical contacts of the second electrical interface includes 32 or more electrical contacts. In some embodiments, the set of electrical contacts of the second electrical interface includes 64 or more electrical contacts. In some embodiments, the catheter handle further includes a first actuator coupled to a first controller, the first actuator configured to move the first controller to control a catheter, coupled to the housing, to move in a first direction and a second direction in a first plane that is aligned with the longitudinal axis; and a second actuator coupled to a second controller, the second actuator configured to move the second controller to control a catheter, coupled to the housing, to move in a third direction and a fourth direction in a second plane aligned with the longitudinal axis, the second plane being orthogonal to the first plane. In some embodiments, the first actuator is configured to move a catheter coupled to the catheter handle in the first plane in an anterior and posterior direction. In some embodiments, the second actuator is configured to move a catheter coupled to the catheter handle in the second plane in a left and right direction. In some embodiments, the first and second actuators are rotatable knobs.

Embodiments of a catheter handle and include other features. In some embodiments, the housing, the first interface, the second interface, and the first and second set of electrical connections are heat sterilizable. In some embodiments, the housing, the first interface, the second interface, and the first and second set of electrical connections are manufactured from materials that can be heat sterilizable without damaging the materials. In some embodiments, the catheter handle is manufactured from metallic and non-metallic materials that can be heat sterilized.

In some embodiments, the catheter handle includes a base configured to support the housing such that the catheter handle can be set on a surface and holds the housing apart from the surface. In some embodiments, the base is formed integral with the housing. In some embodiments, the base is coupled to the housing. In some embodiments, the base comprises a set of electrical controls, the set of electrical controls electrically connected to the first set of electrical connectors. In some embodiments, the set of electrical controls comprises two or more controls. In some embodiments, the set of electrical controls comprises an actuable control button or switch. In some embodiments, the base comprises a distal end and a proximal end, the distal end coupled to the housing and the proximal end configured to be set against a surface. In some embodiments, the base comprises a first base section and a second base section, wherein the first base section includes a proximal end for contacting a surface and a distal end positioned between the proximal end and the housing, and the second base section includes a proximal end for contacting a surface and a distal end positioned between the proximal end and the housing. In some embodiments, the set of electrical controls are positioned on the first base section. In some embodiments, the set of electrical controls are positioned on the first base section and the second base section. In some embodiments, the base is further configured to, when the base is set on a surface, to hold the catheter handle stationary and prevent the catheter handle from rotating around its longitudinal axis or rolling.

Some embodiments of the catheter handle include a rotatable section (“tail”) and a stationary section (“body”), the rotatable section being movable around the longitudinal axis relative to the stationary section. In some embodiments, the catheter handle further includes a base coupled to the stationary section. In some embodiments, the rotatable section is coupled to the first interface such that the rotatable section and the first interface are rotatable together relative to the stationary section. In some embodiments, the catheter handle further includes a first actuator having a proximal portion coupled to the housing, the proximal portion comprising one or more magnets; and a distal portion configured to be coupled to the proximal portion, the distal portion configured to be coupled with the proximal portion to form a rotatable knob, the distal portion comprising one or more magnets arranged to mate with corresponding magnets of the proximal portion to couple the proximal portion to the distal portion. In some embodiments, the proximal portion is configured to mate together with the distal portion when there is a sheet of material disposed between the proximal portion and the distal portion.

The catheter handle can be configured to couple to, and work with, one or more types of catheters. In some embodiments, the catheter handle is configured to work with an intracardiac echography (ICE) catheter, an IVUS catheter, an ablation catheter, and/or a FFR catheter. In some embodiments, the second electrical interface of the catheter handle is configured to be electrically coupled to an intracardiac echography (ICE) catheter. In some embodiments, the second electrical interface is configured to be electrically coupled to two or more types of catheters. In some embodiments, the second electrical interface is configured to be electrically coupled to an intracardiac echography (ICE) catheter, an IVUS catheter, an ablation catheter, an/or a FFR catheter.

In one aspect, a catheter handle includes a housing having a longitudinal axis, the housing having a distal end and a proximal end, a mechanical interface on the distal end for coupling to a catheter, an opening on the proximal end of the housing, an aperture extending from the opening into the housing; a first electrical interface positioned on the proximal end of the housing for electrically connecting to a cable; and a second electrical interface positioned at the distal end of the housing, the second electrical interface having a proximal side facing the aperture and distal side facing the opposite direction, the second electrical interface positioned to be accessible on the proximal side via the aperture, and accessible on the distal side to electrically connect to a catheter coupled to the catheter handle. The first electrical interface further includes a set of electrical contacts, an insulator ring coupled to the housing and positioned around the opening, and a set of electrical contacts located in the insulator ring such that the insulator ring electrically insulates the set of electrical contacts from the housing. The first electrical interface is positioned around the opening. The set of electrical contacts include one or more flat electrically conductive contact pads, one or more pogo pins, two or more electrical contacts (e.g., three, four, five, or six or more electrical connectors). The set of electrical contacts are positioned in a circle on the proximal end of the handle. The catheter handle further includes a set of electrical controls connected to the first electrical interface. The set of electrical controls further include two or more electrical controls each connected to a separate electrical contact of the first electrical interface. The second electrical interface includes a set of electrical contacts that extend from the proximal side of the second electrical interface to the distal side of the second electrical interface. In some aspects, the second electrical interface includes thirty-two or more electrical contacts. In some aspects, the second electrical interface includes sixty-four or more electrical contacts. The housing, the first electrical interface, the second electrical interface, and the first and second set of electrical connections are heat sterilizable without damaging the materials. The second electrical interface is configured to be electrically coupled to an intracardiac echography (ICE) catheter. The second electrical interface is configured to be electrically coupled to two or more types of catheters. In some aspects, the second electrical interface is configured to be electrically coupled to an intracardiac echography (ICE) catheter, and IVUS catheter, an ablation catheter, and a FFR catheter.

The handle further includes a first actuator coupled to a first controller, the first actuator configured to move the first controller to control a catheter, coupled to the housing, to move in a first direction and a second direction in a first plane that is aligned with the longitudinal axis; and a second actuator coupled to a second controller, the second actuator configured to move the second controller to control a catheter, coupled to the housing, to move in a third direction and a fourth direction in a second plane aligned with the longitudinal axis, the second plane being orthogonal to the first plane. The first actuator further includes a proximal portion coupled to the housing, the proximal portion having one or more magnets; and a distal portion configured to be coupled to the proximal portion, the distal portion configured to be coupled with the proximal portion to form a rotatable knob, the distal portion having one or more magnets arranged to mate with corresponding magnets of the proximal portion to couple the proximal portion to the distal portion. The proximal portion is configured to mate together with the distal portion when there is a sheet of material disposed between the proximal portion and the distal portion. The first actuator is configured to move a catheter coupled to the catheter handle in the first plane in an anterior and posterior direction. The second actuator is configured to move a catheter coupled to the catheter handle in the second plane in a left and right direction and is positioned on the housing aligned in the second plane. The first and second actuators are rotatable knobs. The catheter handle is manufactured from metallic and non-metallic materials that can be heat sterilized.

The catheter handle further includes a base configured to support the housing such that the catheter handle can be set on a surface and holds the housing apart from the surface. The base is formed integral with the housing. The base is coupled to the housing. The base further includes a set of electrical controls electrically connected to the first set of electrical connectors, a distal end, and a proximal end. The set of electrical controls includes two or more controls and an actuable control button or switch. The distal end is coupled to the housing and the proximal end configured to be set against a surface. The base further includes a first base section and a second base section, wherein the first base section includes a proximal end for contacting a surface and a distal end positioned between the proximal end and the housing, and the second base section includes a proximal end for contacting a surface and a distal end positioned between the proximal end and the housing. In some aspects, the set of electrical controls are positioned on the first base section. In some aspects, the set of electrical controls are positioned on the first base section and the second base section. The base is further configured to, when the base is set on a surface, to hold the catheter handle stationary and prevent the catheter handle from rotating around its longitudinal axis or rolling.

The housing further includes a rotatable section, a stationary section, and a base. The rotatable section is movable around the longitudinal axis relative to the stationary section. The base is coupled to the stationary section. The rotatable section is coupled to the first interface such that the rotatable section and the first interface are rotatable together relative to the stationary section.

### DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A is a perspective view of an example of an ICE catheter handle 1, illustrating various structural and functional features of some embodiments.

FIG. 1B is a side view of the example of the embodiment of the ICE catheter handle 1 illustrated in FIG. 1, and showing that the handle 1 is configured to be coupled to a cap 10 which is coupled to a catheter 26.

FIG. 2A is an example of a multi-mode catheter system that includes a catheter handle and a control system that can be used with different types of catheters.

FIG. 2B is a perspective view of an example of another embodiment of a durable (reusable) catheter handle, which can be used with an ICE catheter, a IVUS catheter, an RF ablation catheter, and a FFR catheter, in various implementations.

FIG. 2C illustrates a perspective view of a distal end of the embodiment of the catheter handle illustrated in FIG. 2B.

FIG. 2D illustrates a side elevation view of the embodiment of the catheter handle illustrated in FIG. 2B.

FIG. 2E illustrates a cross-sectional side elevation view of the embodiment of the catheter handle illustrated in FIG. 2B.

FIG. 2F illustrates a another cross-sectional side elevation view of the embodiment of the catheter handle illustrated in FIG. 2B.

FIG. 3 illustrates a schematic of the body 18 and the tail 19 portions of the embodiment of the catheter handle illustrated in FIG. 2B showing an example of a frictional coupling between the body 18 and tail 19 portions.

FIG. 4A illustrates a schematic of the body 18 and the tail 19 portions of the embodiment of the catheter handle illustrated in FIG. 2B showing an example of a manually actuated locking pin and lock plate assembly that can be used to lock the rotatable tail 19 in a certain position relative to the body 19.

FIG. 4B illustrates a schematic of the front elevation view of the lock plate 91 illustrated in the embodiment of FIG. 4A.

FIG. 4C illustrates a schematic of the body 18 and the tail 19 portions of the embodiment of the catheter handle illustrated in FIG. 2B showing an example of a solenoid movable locking pin and lock plate assembly that can be used to lock the rotatable tail 19 in a certain position relative to the body 19.

FIG. 5A illustrates a schematic of the body 18 and the tail 19 portions of the embodiment of the catheter handle illustrated in FIG. 2B showing an example of an insertable core 108 shown as being partially inserted into an aperture in the catheter handle, and illustrating an electrical interface of the core, and a corresponding electrical interface on the tail 19 of the catheter handle for providing electrical connections between controls on the catheter handle and the core and connected cable, which when in-use is connected to a control box.

FIG. 5B illustrates an elevation view of the proximal-facing surface 116 of an insulator ring 124, which is part of the electrical interface 64 on the proximal end 20 of the catheter handle to connect to the core proximal electrical interface 110, according to some embodiments.

FIG. 5C illustrates an elevation view of the electrical interface 122 of the distal end of the core that connects to the proximal side 128 of the electrical interface 130 of the distal end 120 of the catheter handle, according to some embodiments.

FIG. 5D illustrates a schematic of the body 18 and a tail 19 portions of the embodiment of the catheter handle illustrated in FIG. 2B showing an example of an insertable core 108 shown inserted into the aperture 4 in the catheter handle and connected to the proximal side of the handle distal electrical interface 126, according to some embodiments.

FIG. 5E illustrates a schematic of an elevation view of the proximal side of the handle distal electrical interface 128, according to some embodiments. and a tail 19 portions of the embodiment of the catheter handle illustrated in FIG. 2B showing an example of an insertable core 108 shown inserted into the aperture 4 in the catheter handle and connected to the proximal side of the handle distal electrical interface 126.

FIG. 5F illustrates a schematic of an elevation view of the distal facing side if the distal electrical interface 130, according to some embodiments.

FIGS. 5G and 5H illustrates an example an actuator that includes a distal portion 67 and a proximal portion 68 which are configured to be magnetically coupled together, FIG. 5G showing the portions 67, 68 configured to mate together with a sheet of plastic disposed there between allowing the actuator to be accessible while the catheter handle is enclosed within a bag or sleeve, and FIG. 5H showing the distal portion 67 and the proximal portion 68 separated.

FIG. 6 is a perspective view of an example of certain internal structure of a catheter handle 1 that is used to mechanically control bending of a catheter in two orthogonal directions and allows the rotation of an internal portion of the catheter while an outer portion maintains its shape, where the internal structure includes a swash plate 32 coupled to a pivot 34, and pushrods 38a-38d that are movable to push against the swash plate 32 to move the swash plate 32.

FIG. 7 is a partial transparent view of an example of internal structure of a catheter handle 1, illustrating details of pushrods 38a-38d, a swash plate 32, and a pivot 34 that allows the swash plate to pivot in two orthogonal directions, a center tube 40, and pullwire anchors 38a-38d.

FIG. 8 illustrates a side view of the embodiment of the catheter handle illustrated in FIG. 2B and a catheter 26 and cap 10 assembly that is coupleable to the catheter handle.

### DETAILED DESCRIPTION OF CERTAIN INVENTIVE ASPECTS

Ever since the invention of the modern disposable catheter in 1940s, it has enjoyed rapid growth and expansion in medical fields. To date, it has found applications in treating cardiovascular, urological, gastrointestinal, neurovascular, and ophthalmic diseases. In treating cardiovascular diseases, there exist at least four catheter modalities, including intracardiac echocardiography (ICE), intravascular ultrasound (IVUS), radiofrequency (RF) ablation, and fractional flow reserve (FFR). Intracardiac echocardiography (ICE) adopts a microscopic ultrasound array to realize direct visualization of anatomical cardiac structures in heart. It greatly helps procedures such as radiofrequency (RF) ablation. Intravascular ultrasound (IVUS) uses a microscopic ultrasonic array to generate images of a blood vessel, i.e., coronary artery. Radiofrequency (RF) ablation applies heat to destroy diseased tissue in order to stop pain. And, fractional flow reserve (FFR) uses a pressure sensor to measure pressure difference across a coronary artery stenosis to determine its effect on oxygen delivery to the heart tissue.

A catheter modality normally comprises two parts, a hardware unit having electronics with software build-in to send control signals, receive and process data for display and operation, and a catheter including a catheter tip, a handle to operate the catheter, and an electrical connector to connect to the hardware unit. Normally, the hardware unit is disposed on a wheeled platform so that the apparatus can be easily transported to different places, for example a procedure room or a storage room. Different modalities are developed to treat different diseases, and even for a single modality there can be different apparatuses developed by different suppliers. The result is that each apparatus its own hardware unit and catheter. And no two of the catheter apparatuses can share hardware or components.

### Illustrative Examples of Catheter Handle-Related Innovations

### Orthogonal Controls

Catheter-based intra-cardiac echocardiography (ICE) is an imaging modality of some similarity to intra-vascular ultrasound (IVUS). ICE is widely used during interventional cardiac procedures to visualize anatomical features, for example, the atrial septum, the aortic valve, pulmonary veins, etc. ICE can also be used to image interventional devices such as ablation catheters and lasso catheters that are used in performing medical procedures on the heart. ICE catheters include an array of ultrasound transducer elements which is used to generate an ultrasound image, for example, a two-dimensional image “slice.” In various applications, the array can be moved to generate information that is processed and can be displayed as a two-dimensional (2D) image or a three-dimensional (3D) image. Such imaging allows collection of information inside the heart and can be used to visualize cardiac structures and blood flow using Doppler imaging.

When an ICE catheter is deployed, its shape may be bent or curved to reach the target of interest, or a point near the target of interest. To move the ICE catheter to the desired imaging area, conventional ICE catheters, and nearly all catheters, have coaxial rings positioned on the handle for steering control, the axis of the rings being aligned with a longitudinal axis of a catheter handle. Control mechanisms that move a catheter tip are generally referred to herein as “steering controls.” ICE catheters accomplish steering control by rotating one ring for steering in an anterior/posterior (up/down, e.g., with respect to the orientation when being used in a patient lying flat), and by rotating the other ring for steering in a left/right direction. The rotation of these rings can be transferred into bending of the catheter shaft, specifically the catheter tip, by pull wires and a system of pulleys. The fundamental challenge of this control method is that the movement of the rings does not logically map to the movement of the catheter. This challenge is tenable when the catheter is visible but becomes significantly more challenging when the catheter is occluded by the body.

Typically, catheter steering for anterior/posterior movement and left/right movement is in two orthogonal planes. An aspect of the disclosed embodiments provides an anterior/posterior steering control and a left/right steering control which are each positioned to align with one of the orthogonal planes through which the catheter tip moves when the corresponding steering control is used to move the tip of the catheter. That is, the configuration or alignment of the steering controls be orthogonally aligned on a catheter handle. For example, a first steering control can be disposed in a first plane coincident with the longitudinal axis of the catheter handle to correspond to posterior/anterior movement along the first plane. A second steering control can be disposed in a second plane that is orthogonal to the first plane and coincident with the longitudinal axis of the catheter handle, and the second control wheel is to correspond to left/right movement along the second plane. The combination of the first posterior/anterior movement in the first plane and the second left/right movement in the second plane allows the tip of the catheter to be steered, bending the catheter tip as needed as it is navigated towards and in a heart. By orienting the controls in the direction of their steering effect, steering control of the catheter in vivo significantly easier for the operator. In various embodiments, the controls can be mechanical and include wheels (e.g., thumbwheels), rotatable dials, and the like. Or the controls can include electric components and include switches, rotatable dials, and other actuators. As used herein, if two things are “orthogonally aligned” it generally means they are aligned 90 degrees or nearly so with respect each other. In some examples, the anterior/posterior control and the left/right control can be orthogonally aligned, or substantially orthogonally aligned on the catheter handle. In some embodiments, “substantially orthogonally aligned” as used herein, can refer to an alignment that of the items at 90 degrees plus or minus about 1, 2, 3, 4, 5, 6, 7, 8, 9, 10, 11, 12, 13, 14, or 15 degrees.

### Durable Catheter Handle

Embodiments herein describes a universal (or multi-mode) reusable (durable) catheter handle platform for surgical interventions with a catheter. The handle can interface with disposable and reusable catheters for a variety of applications. This universality is accomplished through a standardized mating interfaces on the catheters and the handle. The interfaces include connections both electrical and mechanical to accommodate a variety of catheter types and treatment modalities. The handle platform is also capable of connecting to a variety of driving systems such as Ultrasound, RF generators, Electro-anatomical mapping systems etc., via both wired and wireless connections. These connections can be standardized by either a wireless protocol such as Wi-Fi, Bluetooth, Zigbee, 802.11ah, etc., or through a standardized mating connector for wired connections. Universality of a catheter handle platform is clinically advantageous for a number of reasons. The primary advantage is that a universal control interface eliminates the potential for confusion or mis-operation of the control interface due to experience with multiple disparate control interfaces. For example, to complete an atrial fibrillation procedure a cardiologist may need to use an ICE catheter that may have a coaxial ring steering interface in addition to a RFA catheter that may have a knob type single plane steering interface. These both call for different motions to affect the desired steering response at the proximal end of each catheter, yet many ablation procedures call for repeated switching between manipulating these two catheters.

Having a unified interface allows for a consistent repeatable interaction between the catheter and the user. The standardized catheter connection interface includes provisions for all the different types of catheters that can be connected. These provisions can include mechanical connections for steering control of catheter through the handle, fluidic connections for the transmission of fluids such as saline, liquid nitrogen/argon, or drugs, or the remote measurement of pressures. Electrical connections such as RF connections for ultrasonic catheters such as ICE or IVUS, or high voltage connections for RF and Pulse Field ablation. They can also contain digital data connections to various sensor based catheters for applications such as temperature measurement and pressure measurement or video cameras.

The connection interface(s) for driving systems include both wired and wireless connections that are capable of conveying control and data to control systems, power (RF, AC, DC), and fluids if needed in order to support a range of catheter applications and treatment modalities. These interfaces facilitate multidirectional transfer of data and control signals between the handle, the catheter, and the driving system.

Detection and identification of different catheters can be accomplished through electrical connections. These connections can convey catheter presence through simple conductive circuits connected to GPIO pins on an embedded microcontroller in the handle. Identification of catheter types can also be accomplished through conductive circuit patterns connected to an array of GPIO pins, in this case each catheter type presents a different conductive pattern resulting in a unique binary pattern as registered by the digital inputs of the controller. Similarly, this process can be accomplished with fewer connections through analog measurements for example assigning a unique resistance value to each catheter this in turn produces a unique voltage value when connected to a resistor network in the handle. This voltage value can be digitized to correspond to a unique binary pattern in the controller. Furthermore, serial data connection between catheter and handle can facilitate the transfer of more data for catheter identification. In addition to catheter type, device specifics such as serial number, lot number, date of manufacture, etc., or factory characterized performance values such as array sensitivity or bandwidth. This can greatly improve and simplify device traceability for a number of purposes such as recall traceability and blacklisting, counterfeit detection, device lifecycle management, fault and performance logging. This information can be stored in the disposable catheter with a small, low cost memory chip such as FLASH or EEPROM

The handle platform includes many electronic and mechanical user controls spanning a variety of interface types. Mechanical controls can include control of catheter bending or steering in one or more planes, rotation of catheter components such as the outer catheter shaft or a rotating inner lumen, dispensing of fluids. Electrical controls can include buttons, sliders, knobs, touch screen interfaces, etc. These can control devices in the catheter as well as the driving systems that are attached.

The handle platform can contain many mechanisms for disambiguation when multiple catheters are being used. These can include screens allowing for the display of the connected catheter. They can also have multicolor optical indicators to allow software defined color coding of handle devices. The handle platform can also employ non-visual disambiguation through haptic or audio feedback. Haptic feedback disambiguation can be implemented through the assignment of different types of haptic sensations or patterns, for example: a handle connected to an ICE catheter can respond to being picked up by generating a vibration pattern of three short pulses, while connected to an RFA catheter it can generate a single long pulse). Audio feedback can be implemented in the form of distinct sounds emitted corresponding to different catheter handles or in some embodiments the handle can announce the type of the catheter connected to it, this can be triggered through a variety of methods, such as detecting that the handle has been picked up or if a button on the handle is pressed or in response to a verbal query processed by a speech recognition system.

### List of Certain Components

The following is a list of certain components that are described and enumerated in this disclosure in reference to the above-listed figures. However, any aspect of the devices illustrated in the figures, whether or not named out separately herein, can form a portion of various embodiments of the invention and may provide basis for claim limitation relating to such aspects, with or without additional description. Generally, herein, reference to a “distal” portion indicates a portion/component that is positioned closest to the patient when the device/component is in use (e.g., during an intracardiac echocardiography procedure), and reference to a “proximal” portion indicates a portion/component that is positioned farther from the patient when the device/component is in use. The enumerated components include:
1 catheter handle
2 housing
3 catheter system
4 aperture
5 base
6 first actuator (posterior/anterior thumbwheel)
8 second actuator (left/right thumbwheel)
9 interface between body (non-rotatable) portion and tail (rotatable) portion
10 cap (coupled to catheter)
11 distal end of the cap 10
12 proximal end of the cap 10
14 control button
15 control button
16 control button
17 control button
18 body of handle (non-rotatable portion)
19 tail of handle (rotatable portion)
20 proximal end base
21 distal end base
22 first portion of base
24 second portion of base
25 non-rotatable portion of base
26 catheter
28 connector port in handle
29 cable, for providing signals to processing equipment
30 pushrod(s) coupled to an actuator
31 rounded end of pushrod
32 swash plate
33 distal surface, swash plate 32
34 center pivot
36 pullwire feedthrough
38a-d pullwire anchors
40 center tube
41 distal end of catheter
42 locking edge
44 locking recess
46 handle electrical interface
48 cap electrical interface
50 first controller
52 second controller
54 pushrod distal end
56 pushrod proximal end
58 pivot tip of center pivot 34
60 round rod, coupled to swash plate 32
62a-d pullwires
64 electrical interface on handle electrically connected to handle control buttons
65 opening at proximal end of handle connected to aperture in handle
66 actuator shaft
67 distal portion actuator
68 proximal portion of actuator
70 distal end to proximal end axis (longitudinal axis of handle)
72 plane 1
74 plane 2
75 distal surface of tail of handle
76 proximal surface of tail of handle
77 portion of base configured to contact a surface
78 portion of base coupled to housing
79 distal end of base
80 swash plate ring
81 proximal end of base
82a-d wires
87 space between tail and body portion of handle in which O-ring 89 is positioned
88 rotational movement of tail 19 portion of catheter handle around axis 70
89 O-ring
91 lock plate
93 slider to lock tail 19 position relative to body 18 of handle
94 cap assembly of actuator
95 locking pin
96 shaft assembly of actuator
97 locking holes
98 outside surface lock plate
99 inside surface of lock plate
100 lock plate aperture
102 solenoid
104 connection to handle control button (or another control) to control solenoid
108 core
110 core proximal electrical interface
112 pogo pins
114 electrical connections to handle control buttons
116 electrical contact on handle electrical interface 64
118 proximal end of core
120 distal end of core
122 electrical interface core distal end
124 insulator ring
126 handle distal electrical interface
128 proximal side of handle distal electrical interface
130 electrical interface distal side of handle
132 proximal-facing surface of insulator ring 124
134 control system (multi-mode)
136 ICE catheter
138 RF ablation catheter
140 IVUS catheter
142 FFR catheter
144 magnets
146 thin material (sterilized sleeve)

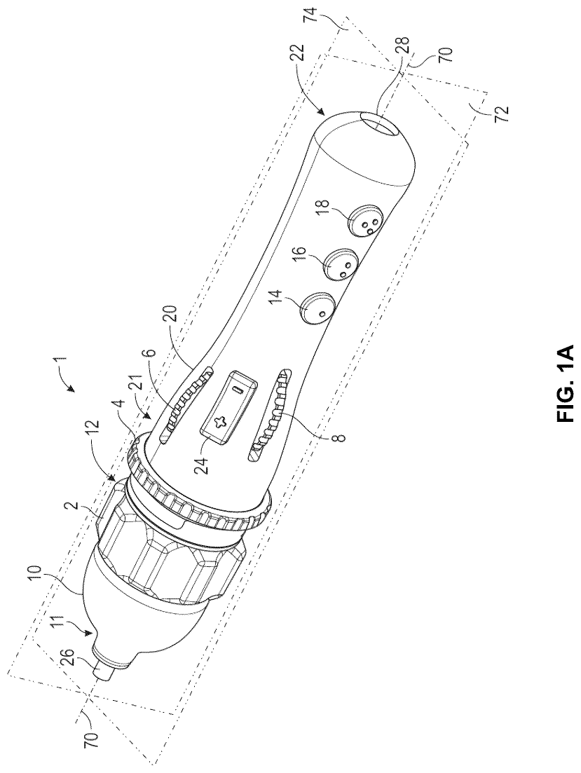

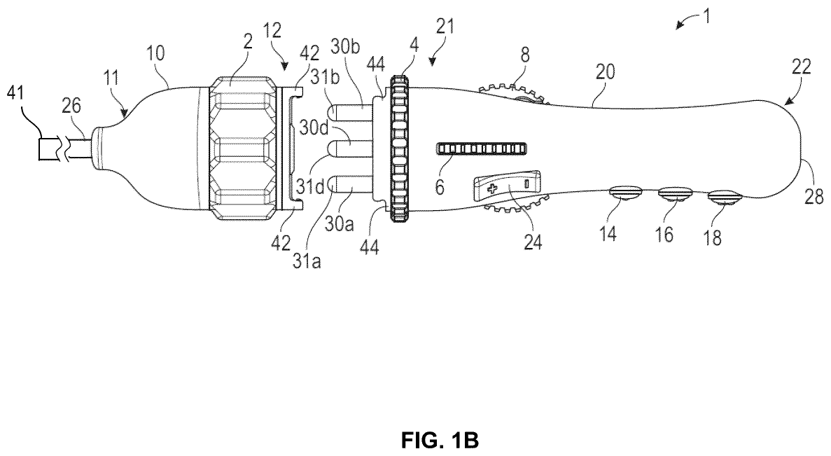

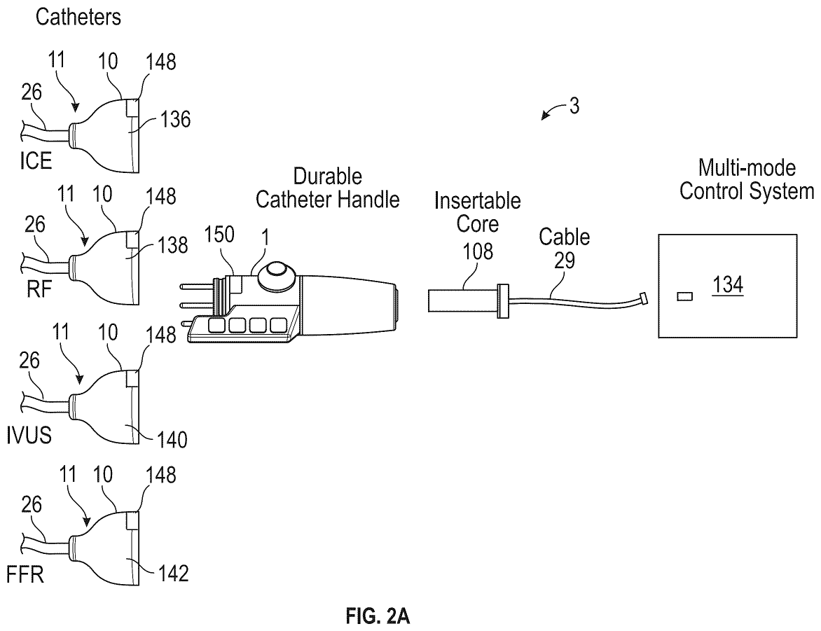

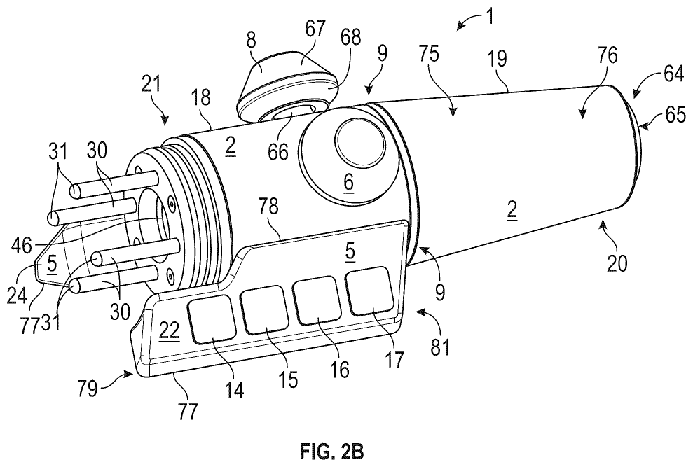

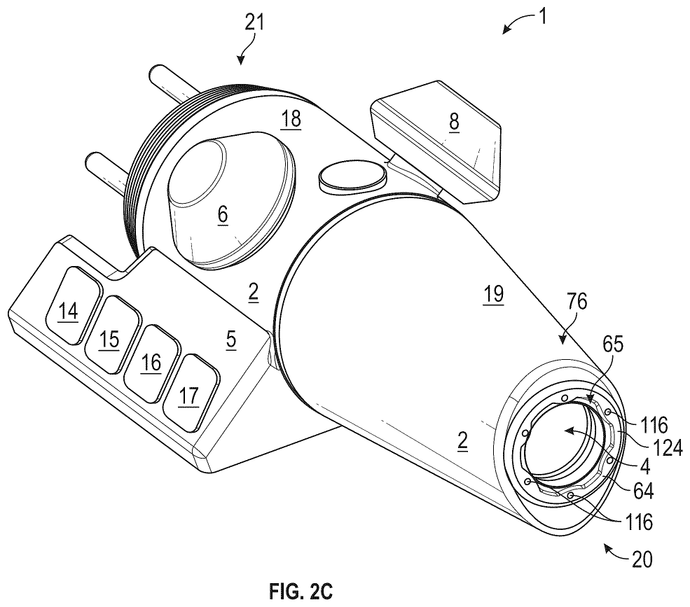

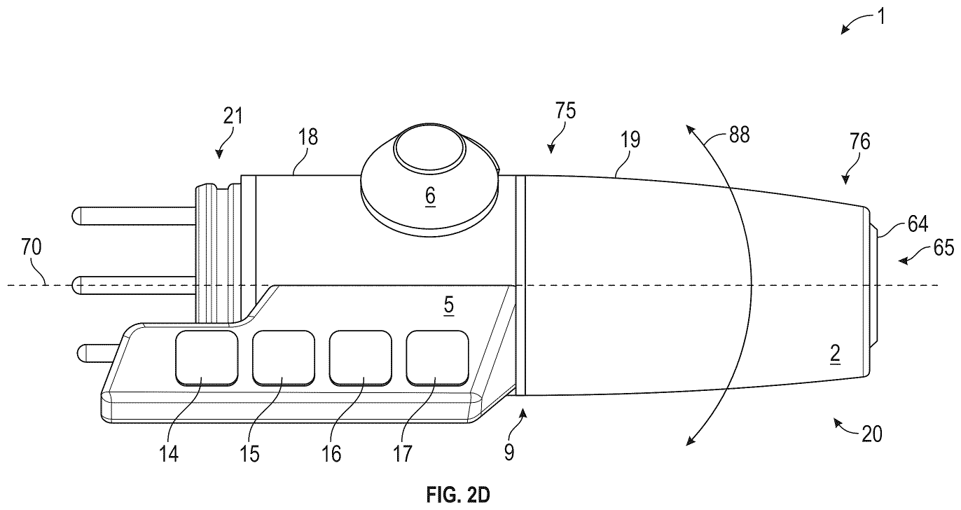

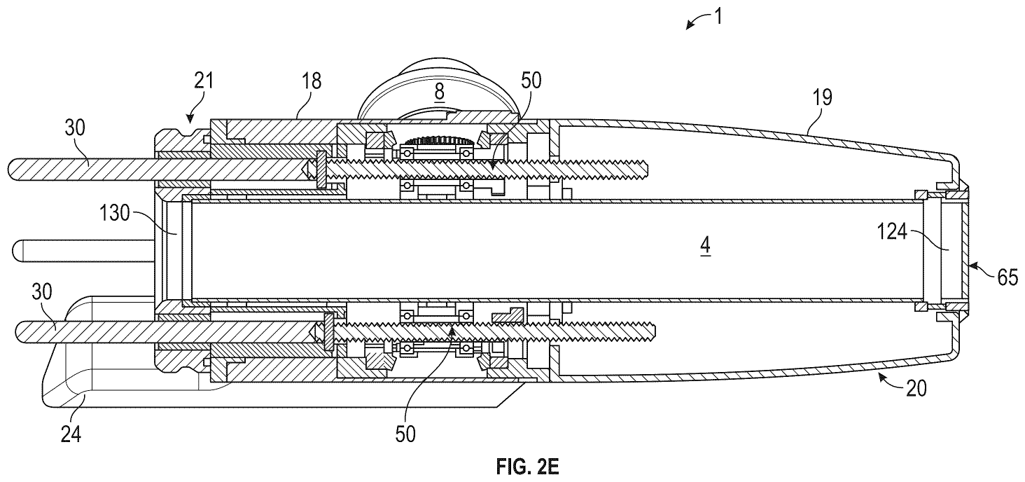

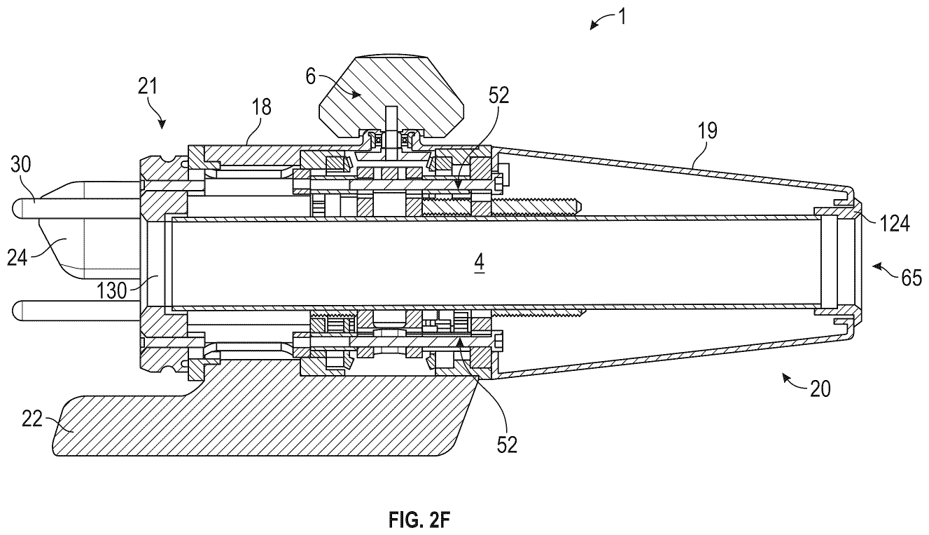

FIGS. 1A and 1B illustrate one embodiment of a catheter handle that may be configured to work with multiple catheters, but can have some limitations for doing so. FIGS. 2A-8 illustrate another embodiment of a catheter handle configured to work with multiple types of catheters, and various structural and functional features of some embodiments. Specifically, FIG. 1A is a perspective view of an example of a catheter handle 1, illustrating various structural and functional features of some embodiments. FIG. 1B illustrates a different view of the catheter handle in FIG. 1A. The illustrated embodiments can be a durable (e.g., reusable) catheter handle. Additional information on the embodiments shown in FIGS. 1A and 1B can be found in U.S. Application Ser. No. 17/820,139, filed Aug. 16, 2022, which is incorporated by reference herein in its entirety.

Although the examples of the catheter handle 1 may sometimes referred to as an ICE catheter handle because the functionality provided by the components of the catheter handle are advantageous for use with an ICE catheter, other types of catheters can also be used with the disclosed examples of catheter handles. In an example, the disclosed handles can be used with a catheter configured to perform intracardiac echocardiography (ICE), a catheter configured to perform intravascular ultrasound (IVUS) catheter, a catheter configured to perform radiofrequency (RF) ablation catheter, or a catheter configured to perform fractional flow reserve (FFR). In addition, the disclosed handles can be used with multi-mode catheters. For example, a catheter that is configured to perform ICE and ablation. In another example, a catheter that is configured to perform RF ablation and FFR. In other example, the catheter handle can be used with a catheter that performs any two or more of ICE, IVUS, ablation, or FFR. A catheter that perform two or more functions can be advantageous as it minimizes the invasiveness of having multiple catheters in the patient's vascular system and the heart.

In the embodiment illustrated in FIG. 1A, the catheter handle 1 includes a cap 10 which is a distal portion of the catheter handle 1, and a handle end 20 which is a proximal portion of the catheter handle 1. The outside surface of the cap 10 is sometimes referred to herein as the cap housing. The outside surface of the handle end 20 is sometimes referred to as the handle housing (or simply housing). Herein, “distal” refers to the portion of the catheter handle 1 that is closest to the catheter 26 (shown only in part) and to the patient when the catheter handle 1 is used in a medical procedure. The cap 10 includes a distal end 11 and a proximal end 12. The handle end 20 also includes a distal end 21 and a proximal end 22. The handle end 20 further includes a first actuator 6 (or thumbwheel 6), and a second actuator 8 (or thumbwheel 8). The handle end can also include one or more controls of various types, for example, a rocker switch 24 and three buttons 14, 16 & 18. The handle end 20 includes a connector port 28 for connecting the catheter handle 1 to computing equipment (e.g., ultrasound processing equipment, a display, and the like). In this example, the catheter handle 1 also includes a rotation collar 2 aligned perpendicular to a longitudinal axis 70 of the catheter handle 1, for example, such that the axis of the rotation collar 2 aligns with the longitudinal axis 70. The rotation collar 2 is coupled to a catheter 26 at the distal end of the catheter. The proximal end 41 of the catheter 26 can include an ultrasound array for generating ICE images. The rotation collar 2 is configured to rotate around the longitudinal axis 70 such that a rotational movement of the rotation collar 2 rotates the catheter 26. The catheter handle 1 also includes a locking ring 4 is positioned on the proximal end 21 of the handle end 20 and perpendicularly aligned to the longitudinal axis 70. The locking ring 4 is configured to rotate around the longitudinal axis to lock a position of the catheter in a certain alignment/position.

In FIG. 1A, the ICE catheter handle 1 has a longitudinal axis 70 coincident with the center line if the catheter 1 and going from the distal end 11 of the cap 10 to the proximal end 22 of the handle end 20. Two planes 72 & 74 that are orthogonal to each other are superimposed to the catheter handle 1 for illustration purpose. The plane 72 is the vertical plane in FIG. 1A, which passes through the first actuator 6 and is coincident with the axis 70 and the mid-plane of the first actuator 6. And the plane 74 is the horizontal plane in FIG. 1A, which passes through the second actuator 8 and is coincident with the axis 70 the mid-plane of the second actuator 8. Since the planes 72 & 74 are orthogonal to each other, the first actuator 6 and the second actuator 8 are also orthogonally disposed in the handle 1.

FIG. 1B is a side view of the example of an ICE catheter handle 1 illustrated in FIG. 1A, showing that the handle 1 is configured to be coupled to a cap 10 which is connected to a catheter 26. The cap 10 may be removably coupled to the handle end 20 such that the cap 10 can be easily separated from, and coupled to, the handle end 20. The handle end 20 includes the locking ring 4 which is configured to rotate around the longitudinal axis 70 to realize locking the handle end 20 with the cap 10. The locking mechanism includes locking edges 40 on the cap 10 and locking recesses 44 on the handle end 20. When the handle end 20 is put together with the cap 10, the locking ring 4 is rotate to engage the locking edges 42 with the locking recesses 44, so that the handle end 20 is firmly connected to the cap 10. To disconnect the handle end 20 from the cap 10, the locking ring 4 is rotated to disengage the locking edge 42 and locking recesses 44.

In this example, the handle end 20 also includes control buttons 14, 16, and 18, as shown in FIG. 1A, that can be used to control various functions of the catheter, including imaging functions. In some embodiments, control buttons 14, 16, and 18 are programmable. The handle end 20 also includes an imaging control rocker switch 24 that controls a zoom feature of an ultrasound array on the catheter. The handle end 20 further includes a connection port 28 to connect the handle end 20, and the catheter attached to the handle end 20, to processing equipment, for example, image processing equipment to process and display ultrasound information generated by the catheter.

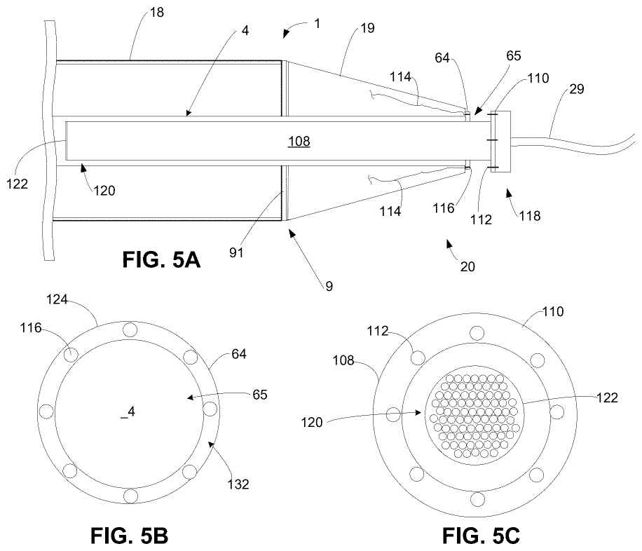

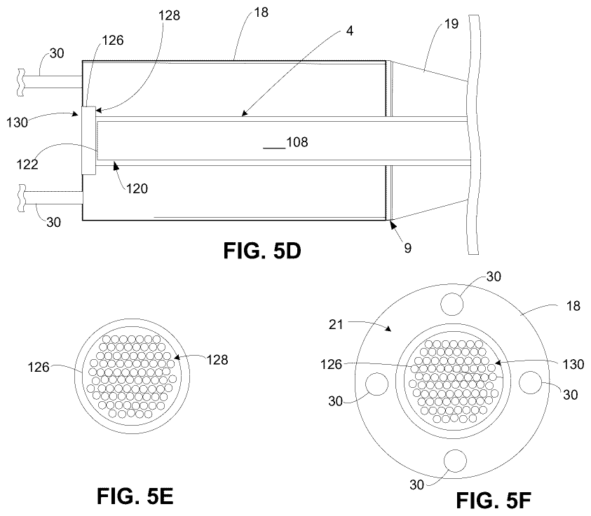

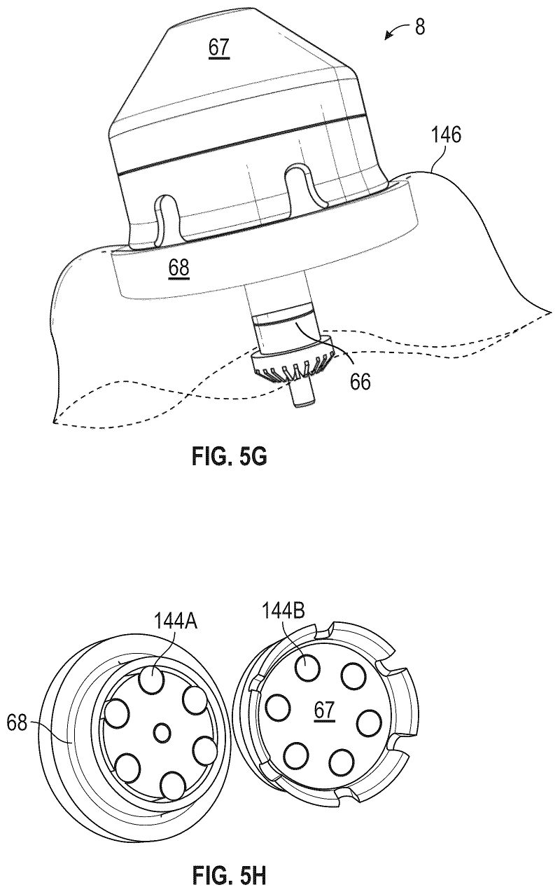

FIG. 2A is an example of an embodiment of a catheter system 3 that includes a catheter handle 1 that can be used with different types of catheters 26, and a multi-mode control system 134 that can be used to process information from different types of catheters. The catheter handle 1 illustrated in FIGS. 2A-8 is a different embodiment than the catheter handle illustrated in FIGS. 1A and 1B, and although some of the features are similar or the same. Referring now to FIG. 2A, the catheter system 3 includes a durable catheter handle 1 which can be mechanically and electrically coupled to a variety of different catheters. For example, an ICE catheter 136, a RF ablation catheter 138, an IVUS catheter 140, or a FFR catheter 142. An insertable core 108 can be mechanically and electrically coupled to the catheter handle 1, by inserting the core 108 into a longitudinal aperture 4 (FIG. 2E) in the catheter handle 1 via an opening 65 (FIG. 2C) on the proximal end of the catheter handle 1. As described in more detail in reference to FIGS. 5A-5E, the core 108 provides electrical connection to control buttons or switches of the handle (e.g., on the base), and provides electrical connections through which signals can be communicated (e.g., received or sent) between a control system 134 and a catheter 26 coupled to the catheter handle 1.

The core 108 connected to a cable 29 which is connectable to a multi-mode control system 134. The control system 134 is configured to communicate with and control a variety of different types of catheters connected to the catheter handle 1, in process signals and information received from a catheter 26 attached to the catheter handle 1. In some embodiments, the control system 134 is configured to automatically determine the type of catheter that is attached to the handle. The control system 134 can then control signals sent to the catheter and process signals received from the catheter based on the determined type of catheter. In some embodiments, the control system is configured to authenticate the catheter attached to the handle. In some embodiments, the control system 134 can use this information to then determine if the catheter is a genuine catheter (instead of a knock-off). In some embodiments, the control system 134 can use information to determine if the catheter being used is subject to a recall or has any other issues with it such that should not be used for the procedure. In an example, the determination of the type of catheter or the authentication of the catheter can be done by the control system by sending certain signals to the catheter during a setup process determining the type of catheter the authentication of the catheter based on signals received back from the catheter. In another example, the determination of the type of catheter or the authentication of the catheter is facilitated by an authentication chip in the catheter which can be queried by the control system 134.

FIG. 2B is a perspective view of an example of an embodiment of a durable (reusable) catheter handle 1, which can be used with an ICE catheter, a IVUS catheter, an RF ablation catheter, and a FFR catheter, or various combinations of an ICE catheter, a IVUS catheter, an RF ablation catheter, and a FFR catheter, in various implementations. FIGS. 2C-2F illustrate other views of this embodiment.

In this example, catheter handle 1 has a housing 2 that has a proximal end 20 and a distal end 21. The catheter handle 1 includes a body portion (“body”) 18 on the distal end 21 and a tail portion (“tail”) 19 on the proximal end 20. The body 18 is coupled to a base 5. The base 5 is structured to prevent the catheter handle 1 from rotating when the catheter handle is set on a surface (e.g., a portion of a patient). In this example, the base 5 includes a first base portion 22 in the second base portion 24. The body 18, being coupled to the base 5, is non-rotatable relative to the base 5.

The catheter handle 1 further includes a first actuator 6 and a second actuator 8 positioned on the body 18 approximately 90 degrees apart. Similar to the actuators 6, 8 in FIG. 1A, the first and second actuators 6, 8 in FIG. 2B are coupled to mechanical assemblies that move pushrods 30 to control the angle of the swash plate 32 in the cap 10, which is connected to the catheter 26. In this embodiment, the first and second actuators 6, 8 are rotating knobs, each controlling a pair of oppositely positioned pushrods 30. Because this embodiment of the catheter handle includes an aperture 4 that runs throughout the housing 2, the mechanical assemblies that are coupled first and second actuator 6, 8 and the pushrods 30 are positioned between the aperture 4 and the outside surface of the housing 2. An example of a mechanical assembly that can be used in this embodiment is illustrated in FIGS. 2E and 2F. As illustrated in FIG. 2B, In this embodiment, each actuator 6, 8 includes an actuator shaft 66 that extends from mechanical assembly positioned inside the housing 2, through the housing 2. The actuator shaft 66 is coupled to a proximal portion 68 of an actuator, and a distal portion 67 of the actuator is coupled to the proximal portion 68. The proximal portion 68 and the distal portion 67 can be removably coupled together, for example, by using magnets. This allows a use case where the catheter handle 1 can be within a sterilized sleeve (or other material) with the proximal portion 68 also within the sterilized sleeve, and the distal portion 67 positioned on the outside of the sleeve and coupled to the proximal portion 68 such that the actuators 6, 8 can be easily used while the catheter handle is within the sterilized sleeve. An example of this option is illustrated in FIG. 5G.

The base 5 includes an upper portion 78 that is coupled to the body 18 and a lower portion 78 that is configured to contact surface and in such a position hold the catheter handle stationary such that the catheter handle 1 as a whole does not rotate. In various configurations of the base 5 are contemplated. In this embodiment, the base 5 includes a first portion (or leg) 22 and a second portion (or leg) 24 that are positioned along the length of the body 18 and extend past the body 18 at the distal end 21 of the catheter handle to provide stability when the catheter handle/base is placed on a surface. Typically, when in use, catheter handle/base is placed on a portion of the patient (for example, a leg of the patient) and the base being configured to have the first portion and the second portion can increase the stability when the surface is not flat (e.g., at least slightly cylindrical). In this embodiment, the base includes one or more control buttons (e.g., control buttons 14-17 in this example) which provide easily accessible controls for catheter procedures. In some embodiments, the functionality of the control button are predetermined, while in other embodiments the functionality of the control buttons can be changed based on the user's preference for the procedure that is being performed.

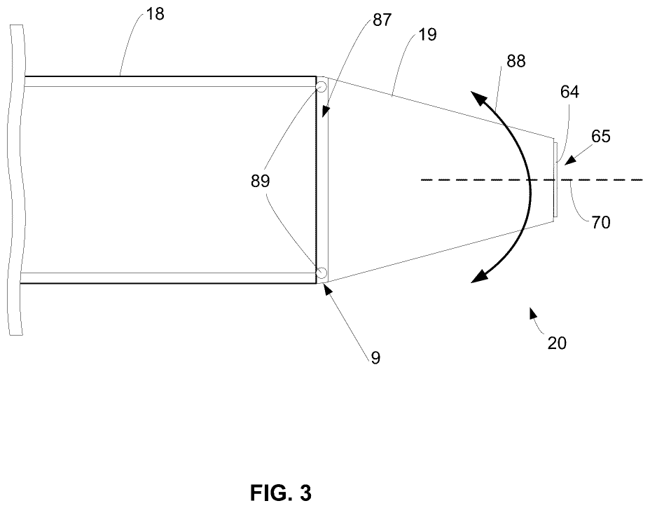

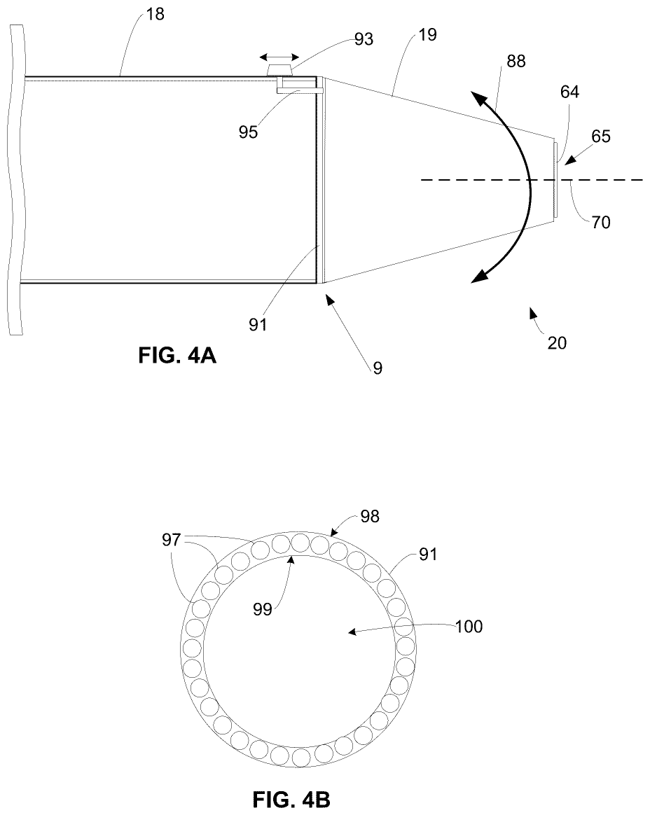

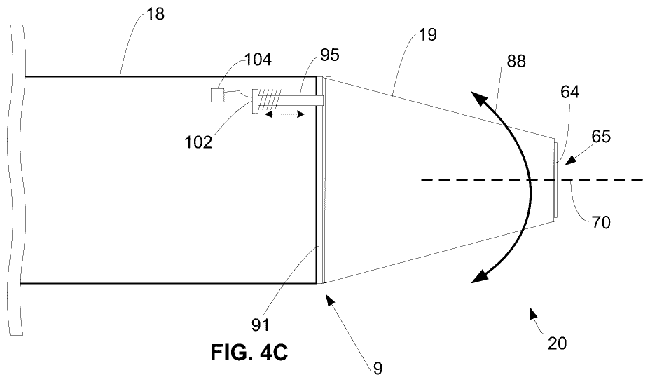

The tail 19 is rotatable, relative to the body 18 and the base 8, around the longitudinal axis 70 (FIG. 3). Typically, the tail 19 is typically rotated to an extent around the longitudinal axis 70, but is not rotated 360°. For example, the tail 19, and corresponding connected catheter, may be rotated in the range of about 0.1-180 degrees clockwise or counterclockwise. Typically, the tail 19 can be rotated between about 1° and 90°, between about 1° and 45°, between about 1° and 30°, between about 1° and 15°, or between about 1° and 7.5° (or less). An interface 9 is positioned between the rotatable tail 19 and the body 18. The interface 9 can include various structures that facilitate controlled movement of the tail 19 relative to the body 18, and locking the position of the tail 19 relative to the body 18, some examples being illustrated in FIGS. 3 and 4A-4C. The tail 19 includes a distal surface 75 and the proximal surface 76. In some embodiments, the distal surface 75 is generally cylindrical. In some embodiments, the proximal surface 76 is generally oval which can facilitate rotation of the tail 19. The tail 19 is coupled to the pushrods 30 and other coupling structure at the distal end 21 of the catheter handle which is used to attach a catheter to the catheter handle. Accordingly, when the tail 19 is rotated around the longitudinal axis 70, the pushrods 30 and other controller mechanisms coupled to the actuators 6, 8, the coupling structure that attaches to the catheter, and a catheter attached to the catheter handle also rotates, while the body 18 and the base 5 do not rotate. The core 108 is mechanically and electrically coupled to the tail 19, and to the catheter, and the core 108 also rotates when the tail 19 is rotated.

The tail 19 includes, at the proximal end 20, an electrical interface 64 which is configured to be electrically connected to an electrical interface of the core 108 (FIG. 2A) when the core 108 is inserted into an internal aperture of the housing 2 via opening 65 on the proximal end 20. The electrical interface 64 is further illustrated in FIGS. 5A-5C. The electrical interface 64 is electrically connected to the control buttons 14-17, and provides for communication of a signal from a control button to the control system 134, the control box 134 then controlling a function (imaging, ablation, sensing, etc.) of a catheter based on the signals communicated from the control button.

FIG. 2C illustrates a perspective view of a distal end 20 of the embodiment of the catheter handle illustrated in FIG. 2B. As illustrated in FIG. 2C, the shape of the proximal surface 76 of the tail 19 is oval-shaped which can make it easier for a user to grasp the tail 19 and rotate it relative to the body 18. FIG. 2C also illustrates opening 65 which is configured to provide access to an aperture 4 which extends through the housing 2 and is configured to receive the core 108. FIG. 2C also illustrates electrical interface 64 positioned around the opening 65, the electrical interface 64 providing electrical connections to the control buttons 14-17. The electrical interface for includes a plurality of contacts 116 which may with corresponding electrical connections of an electrical interface on the core 108.

FIG. 2D illustrates a side elevation view of the embodiment of the catheter handle illustrated in FIG. 2B. This view illustrates tail 19 can rotate 88 around the longitudinal axis 70.

FIG. 2E illustrates a cross-sectional side elevation view of the embodiment of the catheter handle illustrated in FIG. 2B. In FIG. 2E, the aperture 4 is shown extending from the opening 65 to an electrical interface 130 positioned at the distal end of the aperture 4. Electric interface 130 has electrical connections on both its sides (see FIGS. 5D-5F) and provides a connection between the core 108 and a catheter. When the core 108 (FIG. 2A) is inserted through the opening 65 and into the aperture 4, the core 108 extends to the electrical interface 130, and an electrical interface on the distal end of the core 108 can be connected to the proximal side of electrical interface 130. A catheter can be electrically connected to the distal side of electrical interface 130.

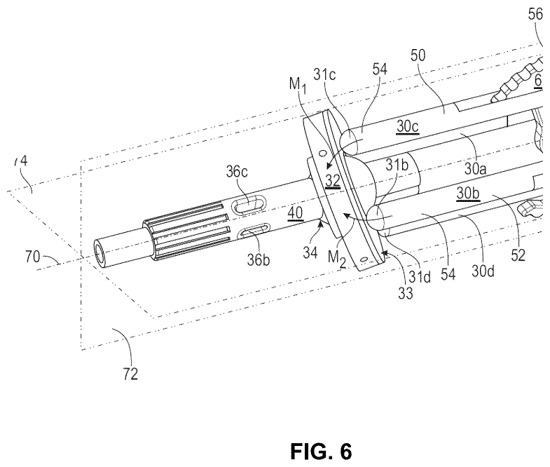

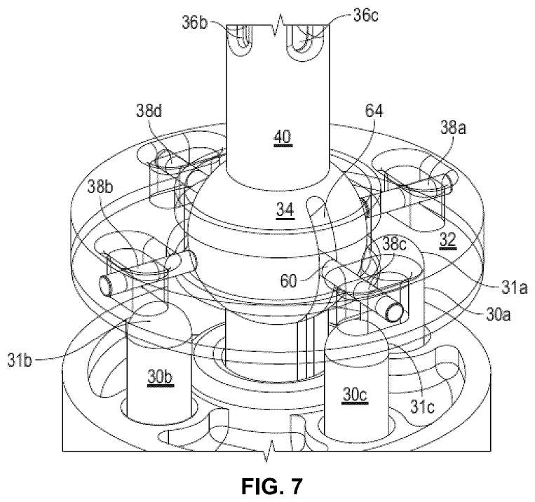

FIG. 2E also illustrates a portion of the mechanical controller 50 the second actuator 8 is coupled to. The controller 50 is also coupled to two oppositely positioned pushrods 30, such that rotation of the second actuator 8 causes the pushrods 30 to extend in a distal direction or retract in a proximal direction to change the tilt of a swash plate 32, which controls steering of the catheter. The interaction between pushrods 30 and the swash plate 32 are further shown in FIGS. 6 and 7. FIG. 2F illustrates another cross-sectional side elevation view of the embodiment of the catheter handle illustrated in FIG. 2B. FIG. 2F illustrates an example of the first actuator 6 being coupled to a mechanical controller 52, where the rotation of the first actuator 6 causes a set of pushrods to either extend and a distal direction or retract and a proximal direction to change the tilt of the swash plate 32.

FIG. 3 illustrates a schematic of the body 18 and the tail 19 portions of the embodiment of the catheter handle illustrated in FIG. 2B showing an example of a configuration that includes frictional coupling of the body 18 and tail 19 at the interface 9. The frictional coupling allows the tail 19 to be rotated to a certain position relative to the body 18, and then the frictional coupling maintain the tail 19 in the position. In this embodiment, the interface 9 includes space 87 between a portion of the body 18 and a portion of the tail 19, and includes an O-ring 89 positioned in the space 87. The tail 19 is attached to the body 18 tightly enough such that the O-ring 89 is at least slightly compressed, such that the interface 9 provides a frictional surface (the O-ring 89) against which the tail 19 can rotate that also holds the tail 19 secure after it has been rotated to the desired position.

FIG. 4A illustrates a schematic of the body 18 and the tail 19 portions of the embodiment of the catheter handle 1 illustrated in FIG. 2B showing an example of a manually actuated locking pin 95 and lock plate assembly 91 that can be used to lock the rotatable tail 19 in a certain position relative to the body 19. In this embodiment, a slider mechanism 93 is located on a portion of the body 18, and is configured to slide a short distance in a direction aligned with a longitudinal axis 70 in a distal or proximal direction. The slider mechanism 93 is coupled to the locking pin 95 which is positioned inside the body 18. The tail 19 includes a lock plate 91 that is positioned in the interface 9. Lock plate 91 is coupled to the tail 19 such that when the tail 19 is rotated the lock plate 91 is also rotated. FIG. 4B illustrates a front elevation view of the lock plate 91 illustrated in the embodiment of FIG. 4A, illustrating its circular configuration. The lock plate 91 includes a plurality of locking holes 97, and the lock plate 91 is positioned in the interface 9 such that the lock holes are aligned with the locking and 95. To rotate the tail 19, slider mechanism 93 moved to a distal position. When the tail 19 is rotated to desired position, the slider mechanism 93 is moved to a proximal position, which moves the locking pin 95 into one of the locking holes 97 which secures the tail 19 in the desired position.

FIG. 4C illustrates a schematic of the body 18 and the tail 19 portions of the embodiment of the catheter handle illustrated in FIG. 2B, showing an example of another embodiment for locking the tail 19 into a desired position relative to the body 18. In this embodiment, instead of being manually operated, a solenoid 102 positioned inside the body 18 can be actuated to move the locking pin 95 engage the lock plate 91. Movable locking pin and lock plate assembly that can be used to lock the rotatable tail 19 in a certain position relative to the body 19. In some embodiments, the solenoid 102 can include an electrical connection 104 can be connected to a control button on the catheter handle (including the base). In some embodiments, the electrical connection 104 is connected directly to a control button on the catheter handle. In some examples, the electrical connection 104 is connected to the electrical interface 64 which can be connected a control button on the catheter handle via the core 108, the cable 29, and the control system 134, such that activating a control button on the catheter handle sends a signal to the control system 134, which in turn sends a control signal via the cable 29, the core 108, and the electrical interface 64 to actuate or de-actuate the solenoid 102.

FIG. 5A illustrates an electrical connection between the catheter handle 1 and a core 108 that can be used to electrically connect controls buttons and/or other electrical features of the catheter handle with a control system 134 (FIG. 2B). Specifically, FIG. 5A shows body 18 and the tail 19 of a catheter handle 1, the aperture 4 running through the central portion of both of the body 18 and tail 19, and a core 108 shown as being partially inserted into the aperture 4. The core 108 is connected to cable 29 that can be connected to the control system 134.

The electrical interface 64 is positioned on the proximal end of the tail 19 around the opening 65. The electrical interface includes an insulator ring 124 and a plurality of electrical contacts 116 in the insulator ring 124. Wires 114 connect the electrical contacts 116 to electrical components in the handle, for example, the control buttons 14-17. FIG. 5B illustrates a view of the proximal-facing surface 116 of an example of the insulator ring 124 and the plurality of electrical contacts 116. When the core 108 is inserted into the aperture 4, an electrical interface 110 on the proximal end of the core 108 electrically connects to the electrical interface 64. An example of the electrical interface 110 is shown in FIG. 5C. The electrical interface 110 includes electrical contacts 112 (e.g., pogo pins) that are arranged to correspond to the electrical contacts 116.

FIG. 5C also illustrates an elevation view of the electrical interface 122 at the distal end of the core 108. In this end view, the electrical interface 110 on the proximal side of the core 108 appears to be around the electrical interface 122. The electrical interface 122 is configured to connect to the proximal side 128 of an electrical interface 126 (FIG. 5D) at the distal end of the aperture 4.

FIG. 5D illustrates a portion of the core 108 positioned in the aperture 4 such that the electrical interface 122 shown in FIG. 5C is connected to the electrical interface 126. The electrical interface 126 includes a proximal side 128 configured to electrically connect to the connecting to the electrical interface 122 of the core 108, and a distal side 130 for connecting to a catheter. The electrical interface 122 includes numerous electrical contacts for connecting to numerous electrical wires of a catheter via the electrical interface 126.

FIG. 5E illustrates an elevation view of an example of the proximal-facing side 128 of the electrical interface 126. FIG. 5F illustrates an example of the distal-facing side 130 of the electrical interface 126. Accordingly, in the example shown herein, a catheter can be electrically connected to the control system 134 via the electrical interface 126 which is at the distal end of the catheter handle, the electrical interface 122 which is at the distal end of the core 108, and the cable 29. In another embodiment (now shown), a catheter handle does not include electrical interface 126, and the catheter can be electrically connected to the control system 134 by connecting the catheter directly to an electrical interface 122 distal end 108.

FIGS. 5G and 5H illustrate an example of an actuator (e.g., actuator 8) that includes a distal portion 67 and a proximal portion 68 which are configured to be magnetically coupled together, the portions 67, 68 configured to mate together with a sheet of material 146 disposed there between allowing the actuator to be accessible within the sterile environment (e.g., of a catheterization laboratory or “cath lab”) while the catheter handle is enclosed behind the material 146 to prevent the catheter handle from being exposed to the cath lab. The material 146 can be, for example, a bag or sleeve that encloses the catheter handle. FIG. 5G illustrates actuator 8 with the distal portion 67 coupled to the proximal portion 68. The actuator 8 includes a shaft 66. When the actuator 8 is coupled to the catheter handle, shaft 66 extends through the housing 2 (see FIG. 2B). FIG. 5H illustrates the sides of the distal portion 67 and the proximal portion 68 that face each other when they are magnetically coupled together. Each of the distal portion 67 and the proximal portion 68 includes one or more magnets 144A, 144B that are arranged to have opposite N/S poles with a corresponding magnet on the other portion such that when the magnets 144A, 144B are positioned near each other they couple distal portion 67 and the proximal portion 68 together, and allow the actuator 8 to be rotated even when the material 146 is positioned between the distal portion 67 and the proximal portion 68

FIG. 6 is a perspective view of an example of certain internal structure of a catheter handle 1 that is used to mechanically control bending of a catheter in two orthogonal directions and allows the rotation of an internal portion of the catheter while an outer portion maintains its shape, where the internal structure includes a swash plate 32 coupled to a pivot 34, and pushrods 38a-38d that are movable to push against the swash plate 32 to move the swash plate 32. The relationship between the four pushrods 30a-30d and the swash plate 32 is more clearly illustrated in FIG. 6, which is an example perspective view of an assembly of some internal parts of the ICE catheter handle 1 shown in FIGS. 1 and 2B. This assembly of internal parts is used to mechanically control the flexing and bending of a proximal end 41 of the catheter 26 which include an ultrasound array which is used to generate ICE images. In FIG. 6 are also established the longitudinal axis 70, and the two orthogonal planes 72 & 74 shown in FIG. 1. According to FIG. 6, the actuator 6 is rotated in such a way to cause the pushrod 30c having a rounded head 31c on the distal end to push against a proximal surface 33 of the swash plate 32, while the pushrod 30d moves away from the swash plate 32. Similarly, the actuator 8 is rotated in such a way to cause the pushrod 30b having a rounded head 31b to push against the proximal surface 33 of the swash plate 32, while the pushrod 30a moves away from the swash plate 32. The rotations of the actuators 6 & 8 can be reversed to cause reversed actions for pushrod pairs 30c & 30d and 30c & 30d. The pushing force from the pushrod 30c to the swath plat 32 causes a moment M1 on the swash plate 32, and the moment M1 is on plane 72 and rotating about the pivot 34, resulting the swath plate to rotate about the pivot 34. Similarly, the pushing force from the pushrod 30b to the swath plat 32 causes a moment M2 on the swash plate 32, and the moment M2 is one plane 74 and rotating about the pivot 34, resulting the swath plate to rotate about the pivot 34. Therefore, the two moments, M1 and M2, are orthogonal to each other. As such, by turning the actuators 6&8 in both directions, the combined effect of the orthogonal moments M1 and M2 is that the swash plate 32 is able to tilt in all different directions, the so called “universal” pivoting.

FIG. 7 is a partial transparent view of an example of internal structure of a catheter handle 1, illustrating details of pushrods 38a-38d, a swash plate 32, and a pivot 34 that allows the swash plate to pivot in two orthogonal directions, a center tube 40, and pullwire anchors 38a-38d. 10 provides a partially transparent perspective view of the assembly of parts of the example illustrated in FIG. 1, including the pushing rods, 30a, 30b, 30c, and 30d, the swash plate 32, the pullwire anchors 38a, 38b, 38c, and 38d, the center tube 40 with the pivot 34, and the pullwire feedthroughs 36b and 36c on the center tube 40. It can be seen clearly that the round rod 60 is coupled with the pivot 34 of the tube 40 through a slotted channel 64, and the swash plate 32 has a rounded hole at its center for the coupling with the pivot 34. Other embodiments can have different types of pivoting mechanisms for tilting the swash plane 32. It is within the scope of the present invention if orthogonal forces or moments are involved to deliver movements to the remote end of the catheter.

Tilting of the swash plate 32 can be determined by different arrangements of pushrods, for example, four pushrods 30a-30d, two positioned in each of the planes 72&74 as a pair. Some other embodiments may include three pushrods to cause orthogonal tilting of the swash plate 32. In some embodiments, the first and second actuators 6, 8 are controls that are electrically coupled to orthogonally steer the catheter. In some embodiments the two actuators may be located at different locations on the handle end 20, including the proximal end 21, the distal end 22, and the middle portion between the proximal end 21 and the distal end 22, or on one side, or on different sides. In some embodiments, each of the orthogonally aligned actuators is a rotatable dials that are mechanically coupled to the pushrods, and move the pushrods toward the swashplate when they are rotated in a first direction and away from the swashplate when they are rotated in a second direction, opposite the first direction. In such embodiments, the actuators can be coupled to the pushrod by one or more gears that translate the rotational motion of the actuator to a longitudinal motion of the pushrod (e.g., similar to a mechanism used in a stepper motor system). In some embodiments, the movement of the actuators can control a stepper motor that moves the pushrod towards and away from the swashplate (depending on the movement of the actuator) in a longitudinal direction. Use of a stepper motor may control the speed of the movement of pushrod and correspondingly the steering movement of end of the catheter, allowing very fine movements to be made with precision. In some embodiments with stepper motors. In various embodiments, the movement of the pushrods 30a-30d may be controlled via the actuators along with a number of other mechanisms, for example, rack and pinion gear drives, or pin and slot joints, and the like.

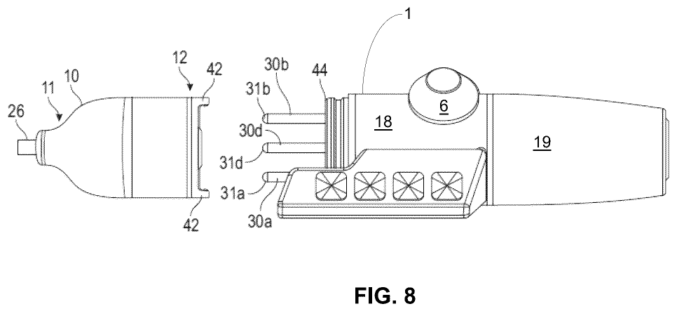

FIG. 8 illustrates a side view of the embodiment of the catheter handle illustrated in FIG. 2B and a catheter 26 and cap 10 assembly that is coupleable to the catheter handle. As shown FIG. 8, a plan view of an example embodiment of the catheter handle 1 of FIG. 2A, the cap 10 may be removably coupled to the distal end of the handle 1 such that the cap 10 can be easily separated from, and coupled to, the handle 1. When the handle 1 is coupled to the cap 10, with the cap 10, rotation of the tail 19 around the longitudinal axis 70 (FIG. 3) rotates the cap 10 and the catheter 26.

In this example, the base 5 includes control buttons 14-17 that can be used to control various functions of the catheter, including imaging functions. In some embodiments, control buttons 14-17 are programmable. When the cap 10 is coupled to the handle, the catheter 26 is electrically connected to the distal side 130 of electrical interface 126. The electrical interface 122 on the distal end 120 of the core 108 is electrically connected to the proximal side 128 of the electrical interface 126 when the core 108 is fully inserted into the aperture 4, which allows signals from the catheter (e.g., ultrasound signals) to be communicated from an array on the catheter 26 to a control/processing module via the core 108 and the cable 29.

### EXAMPLE EMBODIMENTS

In a first embodiment, a catheter handle includes a housing having a longitudinal axis, the housing having a distal end and a proximal end, a mechanical interface on the distal end for coupling to a catheter, an opening on the proximal end of the housing, an aperture extending from the opening into the housing; a first electrical interface positioned on the proximal end of the housing for electrically connecting to a cable; and a second electrical interface positioned at the distal end of the housing, the second electrical interface having a proximal side facing the aperture and distal side facing the opposite direction, the second electrical interface positioned to be accessible on the proximal side via the aperture, and accessible on the distal side to electrically connect to a catheter coupled to the catheter handle. The first electrical interface further includes a set of electrical contacts, an insulator ring coupled to the housing and positioned around the opening, and a set of electrical contacts located in the insulator ring such that the insulator ring electrically insulates the set of electrical contacts from the housing. The first electrical interface is positioned around the opening. The set of electrical contacts include one or more flat electrically conductive contact pads, one or more pogo pins, two or more electrical contacts (e.g., three, four, five, or six or more electrical connectors). The set of electrical contacts are positioned in a circle on the proximal end of the handle. The catheter handle further includes a set of electrical controls connected to the first electrical interface. The set of electrical controls further include two or more electrical controls each connected to a separate electrical contact of the first electrical interface. The second electrical interface includes a set of electrical contacts that extend from the proximal side of the second electrical interface to the distal side of the second electrical interface. In some aspects, the second electrical interface includes thirty-two or more electrical contacts. In some aspects, the second electrical interface includes sixty-four or more electrical contacts. The housing, the first electrical interface, the second electrical interface, and the first and second set of electrical connections are heat sterilizable without damaging the materials. The second electrical interface is configured to be electrically coupled to an intracardiac echography (ICE) catheter. The second electrical interface is configured to be electrically coupled to two or more types of catheters. In some aspects, the second electrical interface is configured to be electrically coupled to an intracardiac echography (ICE) catheter, and IVUS catheter, an ablation catheter, and a FFR catheter.

The handle further includes a first actuator coupled to a first controller, the first actuator configured to move the first controller to control a catheter, coupled to the housing, to move in a first direction and a second direction in a first plane that is aligned with the longitudinal axis; and a second actuator coupled to a second controller, the second actuator configured to move the second controller to control a catheter, coupled to the housing, to move in a third direction and a fourth direction in a second plane aligned with the longitudinal axis, the second plane being orthogonal to the first plane. The first actuator further includes a proximal portion coupled to the housing, the proximal portion having one or more magnets; and a distal portion configured to be coupled to the proximal portion, the distal portion configured to be coupled with the proximal portion to form a rotatable knob, the distal portion having one or more magnets arranged to mate with corresponding magnets of the proximal portion to couple the proximal portion to the distal portion. The proximal portion is configured to mate together with the distal portion when there is a sheet of material disposed between the proximal portion and the distal portion. The first actuator is configured to move a catheter coupled to the catheter handle in the first plane in an anterior and posterior direction. The second actuator is configured to move a catheter coupled to the catheter handle in the second plane in a left and right direction and is positioned on the housing aligned in the second plane. The first and second actuators are rotatable knobs. The catheter handle is manufactured from metallic and non-metallic materials that can be heat sterilized.

The catheter handle further includes a base configured to support the housing such that the catheter handle can be set on a surface and holds the housing apart from the surface. The base is formed integral with the housing. The base is coupled to the housing. The base further includes a set of electrical controls electrically connected to the first set of electrical connectors, a distal end, and a proximal end. The set of electrical controls includes two or more controls and an actuable control button or switch. The distal end is coupled to the housing and the proximal end configured to be set against a surface. The base further includes a first base section and a second base section, wherein the first base section includes a proximal end for contacting a surface and a distal end positioned between the proximal end and the housing, and the second base section includes a proximal end for contacting a surface and a distal end positioned between the proximal end and the housing. In some aspects, the set of electrical controls are positioned on the first base section. In some aspects, the set of electrical controls are positioned on the first base section and the second base section. The base is further configured to, when the base is set on a surface, to hold the catheter handle stationary and prevent the catheter handle from rotating around its longitudinal axis or rolling.

The housing further includes a rotatable section, a stationary section, and a base. The rotatable section is movable around the longitudinal axis relative to the stationary section. The base is coupled to the stationary section. The rotatable section is coupled to the first interface such that the rotatable section and the first interface are rotatable together relative to the stationary section.

### Implementation Consideration

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Headings are included herein for reference and to aid in locating various sections. These headings are not intended to limit the scope of the concepts described with respect thereto. Such concepts may have applicability throughout the entire specification.

Many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

It will also be understood that, when a feature or element (for example, a structural feature or element) is referred to as being “connected”, “attached” or “coupled” to another feature or element, it may be directly connected, attached, or coupled to the other feature or element or intervening features or elements may be present. In contrast, when a feature or element is referred to as being “directly connected,” “directly attached” or “directly coupled” to another feature or element, there may be no intervening features or elements present. Although described or shown with respect to one embodiment, the features and elements so described or shown may apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments and implementations only and is not intended to be limiting. For example, as used herein, the singular forms “a,” “an” and “the” may be intended to include the plural forms as well, unless the context clearly indicates otherwise. It will be further understood that the terms “comprises” and/or “comprising,” when used in this specification, specify the presence of stated features, steps, operations, processes, functions, elements, and/or components, but do not preclude the presence or addition of one or more other features, steps, operations, processes, functions, elements, components, and/or groups thereof. As used herein, the term “and/or” includes any and all combinations of one or more of the associated listed items and may be abbreviated as “/.”

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

Spatially relative terms, such as “forward,” “rearward,” “under,” “below,” “lower,” “over,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features due to the inverted state. Thus, the term “under” may encompass both an orientation of over and under, depending on the point of reference or orientation. The device may be otherwise oriented (rotated 90 degrees or at other orientations) and the spatially relative descriptors used herein interpreted accordingly. Similarly, the terms “upwardly,” “downwardly,” “vertical”, “horizontal” and the like may be used herein for the purpose of explanation only unless specifically indicated otherwise.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

Although various illustrative embodiments have been disclosed, any of a number of changes may be made to various embodiments without departing from the teachings herein. For example, the order in which various described method steps are performed may be changed or reconfigured in different or alternative embodiments, and in other embodiments one or more method steps may be skipped altogether. Optional or desirable features of various device and system embodiments may be included in some embodiments and not in others. Therefore, the foregoing description is provided primarily for the purpose of example and should not be interpreted to limit the scope of the claims and specific embodiments or particular details or features disclosed.

The examples and illustrations included herein show, by way of illustration and not of limitation, specific embodiments in which the disclosed subject matter may be practiced. As mentioned, other embodiments may be utilized and derived therefrom, such that structural and logical substitutions and changes may be made without departing from the scope of this disclosure. Such embodiments of the disclosed subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve an intended, practical, or disclosed purpose, whether explicitly stated or implied, may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The disclosed subject matter has been provided here with reference to one or more features or embodiments. Those skilled in the art will recognize and appreciate that, despite of the detailed nature of the example embodiments provided here, changes and modifications may be applied to said embodiments without limiting or departing from the generally intended scope. These and various other adaptations and combinations of the embodiments provided here are within the scope of the disclosed subject matter as defined by the disclosed elements and features and their full set of equivalents.

1-44. (canceled)

45. A catheter handle platform, comprising:
a catheter handle including
a housing having a longitudinal axis, a distal end, and a proximal end;
a mechanical interface on the distal end of the housing, the mechanical interface configured for coupling to a catheter;
an electrical interface on the distal end of the housing, the electrical interface configured for coupling to a catheter;
a set of electrical controls configured to control devices of a catheter coupled to the electrical interface, the set of electrical controls including two or more electrical controls;
a first rotatable knob positioned on the housing and coupled to a first controller, the first rotatable knob configured to rotate in a first plane that is aligned with the longitudinal axis of the handle, the first rotatable knob configured to move the first controller to control a catheter, coupled to the mechanical interface, to move in a first direction and a second direction aligned with the first plane; and
a second rotatable knob positioned on the housing and coupled to a second controller, the second rotatable knob configured to rotate in a second plane that is aligned with the longitudinal axis of the handle, the second rotatable knob configured to move the second controller to control a catheter, coupled to the mechanical interface, to move in a third direction and a fourth direction aligned with the second plane; and
a base coupled to the housing on a side of the housing opposite the first and second rotatable knobs, the base having an upper portion coupled to the housing and a lower portion configured to be placed in contact with a surface and hold the catheter handle stationary and hold the housing apart from the surface.

46. The catheter handle platform of claim 45, wherein the second plane is orthogonal to the first plane.

47. The catheter handle platform of claim 45, wherein the catheter handle is a reusable durable handle.

48. The catheter handle platform of claim 45, wherein the first rotatable knob and the second rotatable knob are positioned substantially orthogonal to each other.

49. The catheter handle platform of claim 45, wherein the second electrical interface is configured to be electrically coupled to two or more types of catheters.

50. The catheter handle of claim 45, wherein the electrical interface is configured to be electrically coupled to an intracardiac echography (ICE) catheter, an intravascular ultrasound (IVUS) catheter, an ablation catheter, and a fractional flow reserve (FFR) catheter.

51. The catheter handle platform of claim 45, wherein the set of electrical controls are positioned on the base.

52. The catheter handle platform of claim 45, wherein the set of electrical controls comprises an actuable control button.

53. The catheter handle platform of claim 45, wherein the set of electrical controls comprises an actuable switch.

54. The catheter handle platform of claim 45, further comprising the catheter, wherein the catheter is configured for intracardiac echography (ICE), an intravascular ultrasound (IVUS), ablation, or fractional flow reserve (FFR).

55. The catheter handle platform of claim 45, wherein the first rotatable knob is configured to move the catheter coupled to the catheter handle in the first plane in an anterior and posterior direction.

56. The catheter handle platform of claim 55, wherein the second actuator is configured to move the catheter coupled to the catheter handle in the second plane in a left and right direction.

57. The catheter handle platform of claim 45, wherein the housing, the electrical interface, the mechanical interface, and the set of electrical controls are configured to be heat sterilizable.

58. The catheter handle platform of claim 57, wherein the housing, the electrical interface, the mechanical interface, and the set of electrical controls are manufactured from materials that can be heat sterilizable without damaging the materials.

59. The catheter handle platform of claim 45, wherein the base is formed integral with the housing.

60. The catheter handle platform of claim 45, further comprising a base configured to support the housing such that the catheter handle can be set on a surface and holds the housing apart from the surface.

61. The catheter handle platform of claim 45, wherein the base is formed integral with the housing.

62. The catheter handle platform of claim 45, wherein the housing further includes a rotatable section on the proximal end of the housing and movable around the longitudinal axis of the housing, the rotatable section coupled to the mechanical interface such that rotation of the rotatable section around the longitudinal axis correspondingly rotates the mechanical interface around the longitudinal axis.

63. The catheter handle platform of claim 45, wherein the first and second rotatable knobs each comprise:
a proximal portion coupled to the housing, the proximal portion comprising one or more magnets, and
a distal portion configured to be coupled to the proximal portion, the distal portion configured to be coupled with the proximal portion to form a rotatable knob, the distal portion comprising one or more magnets arranged to mate with corresponding magnets of the proximal portion to couple the proximal portion to the distal portion.

64. The catheter handle platform of claim 45, wherein the distal portion and the proximal portion that are configured to magnetically couple together with a sterile barrier placed in-between the distal portion and the proximal portion.
